(19) United States
(12) Patent Application Publication
(10) Pub. No.: US 2007/0048383 A1
(43) Pub. Date: Mar. 1, 2007

(54) Self-assembled endovascular structures

(76) Inventor: Michael N. Helmus, Worcester, MA (US)

Correspondence Address: MAYER & WILLIAMS PC, 251 NORTH AVENUE WEST, 2ND FLOOR, WESTFIELD, NJ, 07090, US

(21) Appl. No.: 11/211,809

(22) Filed: Aug. 25, 2005

(51) Int. Cl. A61K 9/14

(52) U.S. Cl. 424/489; 977/906

(57) ABSTRACT

The present invention is directed to the formation of structures in situ through the principles of ligand binding. These structures are efficacious, for example, for tissue repair as well as for short- and long-term disease and condition management. According to one aspect of the invention, an injectable composition comprising self-assembling nanoparticles is provided. The self-assembling nanoparticles include: (a) a nanoparticle portion, (b) tissue binding ligands attached to the nanoparticle portion, which cause preferential binding and accumulation of the nanoparticles at one or more targeted tissue locations upon injection of the composition into the body, and (c) first and second interparticle binding ligands attached to the nanoparticle portion, which cause interparticle binding upon injection of the composition into the body.

### FIELD OF THE INVENTION

This invention relates to self-assembled endovascular structures, which are useful for the treatment of a variety of diseases and conditions.

### BACKGROUND OF THE INVENTION

The present state of the art concerning the deployment and placement of endovascular medical devices is challenged by profile and delivery issues. For example, before a stent is delivered and positioned appropriately, the specific target site must first be detected and characterized using complex imaging and sensing procedures. This is then followed by cumbersome tracking and delivery procedures using guidewires, catheters, and various other devices. These procedures often have to be repeated to achieve the desired result. Hence, there is great expense associated with these procedures.

Moreover, therapeutic devices capable of intervention at the molecular level, such as Boston Scientific's paclitaxel-based drug-eluting stent, have shown significant potential for disease management. However, while effective, such devices are complex and expensive.

### SUMMARY OF THE INVENTION

The above and other challenges have intensified the need for inexpensive endovascular constructs that are specific, efficacious, and able to be deployed with a high degree of freedom and precision.

In this regard, the present invention is directed to the formation of endovascular structures in situ through the principles of ligand binding. These structures are efficacious, for example, for tissue repair as well as for short- and long-term disease management.

According to one aspect of the invention, an injectable composition comprising self-assembling nanoparticles is provided. The self-assembling nanoparticles include: (a) a nanoparticle portion, (b) tissue binding ligands attached to the nanoparticle portion, which cause preferential binding and accumulation of the nanoparticles at one or more targeted tissue locations upon injection of the composition into the body, and (c) first and second interparticle binding ligands attached to the nanoparticle portion, which cause interparticle binding upon injection of the composition into the body.

Specific examples of applications of the present invention include the in situ formation of endovascular patches for vulnerable plaque and aneurysmal management, expandable stents for increasing blood flow and maintaining vessel patency, contractible patches for congestive heart failure, drug delivery structures, and scaffolding for tissue engineering. However, the application of this platform technology is ubiquitous and many other applications will immediately become apparent to those of ordinary skill in the art upon reading the detailed description and claims to follow.

### DETAILED DESCRIPTION

A more complete understanding of the present invention is available by reference to the following detailed description of numerous aspects and embodiments of the invention. The detailed description of the embodiments which follows is intended to illustrate but not limit the invention.

All publications, patents and patent applications cited herein, whether supra or infra, are hereby incorporated by reference in their entirety.

In accordance with a first aspect of the invention, compositions are provided that contain self-assembling nanoparticles. These nanoparticles, in turn, comprise the following: (a) a nanoparticle portion, (b) tissue binding ligands attached to the nanoparticle portion, which result in the preferential binding and accumulation of the nanoparticles at one or more target locations in the body, and (c) first and second interparticle binding ligands attached to the nanoparticle, which preferentially bind to one another, wherein the first and second interparticle binding ligands can be the same or different.

Compositions in accordance with the present invention may be injected via various routes including intravascular injection (e.g., intravenous injection, intraarterial injection, intracoronary injection, intracardiac injection, etc.), intramuscular injection, subcutaneous injection, and intraperitoneal injection routes, among others. Injection may proceed via various known medical devices including syringes, venous drug delivery catheters, arterial drug delivery catheters, and so forth. Drug deliver catheters are advantageous in certain embodiments as they facilitate more localized, less systemic, drug delivery. Various drug delivery catheter designs are known, including perfusion catheters, injection catheters, and double balloon catheters, among others.

In some embodiments, the nanoparticles are stored or rehydrated with a solution that inhibits binding between the interparticle binding ligands prior to injection. In these embodiments the nanoparticles are injected at concentrations that are low enough to prevent substantial aggregation at the time injection, with the majority of the binding occurring when the nanoparticles come into close association with each other at the assembly site (e.g., due to the presence of the tissue binding ligands on the microparticles). In other embodiments, at least one of the first and second interparticle binding ligands is activated in vivo at the one or more target locations within the body. These features of the invention allow self-assembly of nanoparticles at the target site, while at the same time avoiding premature interparticle aggregation, e.g., prior to injection.

With respect to embodiments in which the interparticle binding ligands are activated in vivo, such activation may proceed via any suitable process. For example, one or both of the interparticle binding ligands may be inactivated by reversibly attaching the same to an inactivating moiety (e.g., a hydrophilic polymer chain, among many other choices) that prevents the interparticle binding ligands from binding to one another. The inactivating moiety is then cleaved from the ligand(s) in vivo at the one or more target locations within the body, for instance, by exposure to enzymes or to light (e.g., using a catheter) to release the inactivating moiety. See, e.g., Subr V, et al. “Release of macromolecules and daunomycin from hydrophilic gels containing enzymatically degradable bonds.” J Biomater Sci Polym Ed. 1990; 1(4):261-78.

Analogously, one or both of the interparticle binding ligands may be inactivated by reversibly attaching the same to an inactivating moiety via a linkage that is thermally cleavable. (In these and other instances herein, the temperatures used are typically sufficiently low to avoid disruption of the linkage between the tissue binding ligands and the tissue at the target locations.) The inactivating moiety is then cleaved from the ligand in vivo by heat (e.g. by heating with MRI, etc., or flushing the area via catheter with a warm solution) to release the inactivating moiety. Linkages which are thermally unstable include metal coordination bonding, for instance, the linkage of acrylamide polymers to histidine groups through metal coordination bonding (See, e.g., Chen et al. and Wang et al. below). Other examples are linkages between groups that pair to one another via multiple hydrogen bonds. Several examples of such molecules are described in Sherrington D C and Taskinen K A, “Self-assembly in synthetic macromolecular systems via multiple hydrogen bonding interactions,” Chem. Soc. Rev., 2001, 30 (2), 83-93, and include the familiar hydrogen bonding between thymine/uracil and adenine and between cytosine and guanine, as well as higher order bonding such as bonding via four hydrogen bonds using uredopyrimidinone residues. (Note that uredopyrimidinone residues bind to one another and thus represent ligands that are the same and yet bind to one another. In this regard, such ligands are also used for interparticle binding in certain embodiments of the invention.)

In other embodiments, one or both of the interparticle binding ligands are embedded within a hydrogel polymer. Upon a triggering event, which makes the hydrogel polymer go from a more hydrophobic to a more hydrophilic state (which is also accompanied by swelling of the hydrogel) or which makes the hydrogel polymer go from a more hydrophilic state to a more hydrophobic state, the binding ligand may be expelled/released from the hydrogel into the biological milieu. For example, hydrogels are known that become more hydrophilic based on changes in pH, osmolality or temperature, upon application of an electric field, and so forth. See, e.g., Chatterjee, et al., Nanotech 2003 Vol. 1, Technical Proceedings of the 2003 Nanotechnology Conference and Trade Show, Volume 1, Chapter 7: Bio Micro Systems, “Electrically Triggered Hydrogels: Mathematical Models and Simulations,” pp. 130-133; Eichenbaum G M, et al. “pH and Ion-Triggered Volume Response of Anionic Hydrogel Microspheres.” Macromolecules. 1998 Jul. 28; 31(15):5084-93; Chen et al., “Responsive hybrid hydrogels with volume transitions modulated by a titin immunoglobulin module.” Bioconj. Chem. 2000 September-October; 11(5): 734-40; Coughlan D C, et al., “Effect of drug physicochemical properties on swelling/deswelling kinetics and pulsatile drug release from thermoresponsive poly(N-isopropylacrylamide) hydrogels.” J Control Release. 2004 Jul. 23; 98(1):97-114; Molinaro G, et al. “Biocompatibility of thermosensitive chitosan-based hydrogels: an in vivo experimental approach to injectable biomaterials.” Biomaterials. 2002 July; 23(13):2717-22. Wang C, et al. “Hybrid hydrogels cross-linked by genetically engineered coiled-coil block proteins.” Biomacromolecules. 2001 Fall; 2(3):912-20. Hence, using the above hydrogel polymers as well as other currently available polymer matrices, ligand release may be triggered, for example, by local pH change (e.g., by flushing the area with an acidic or basic solution via catheter) to ionize ionic groups in the polymer, by heating (e.g. by heating with MRI or flushing the area with a warm solution via catheter) to force a transformation of the polymer beyond a critical transition that allows hydration, or by hydrolysis/enzymatic cleavage to expose hydrophilic groups in the polymer. In addition to (or as an alternative to) ligand coverage and exposure, such triggerable hydrogels may also be used to retain and release drugs.

Activation of the ligand via conformation changes may also be employed, e.g., by denaturation, pH change, temperature change, and so forth.

In certain embodiments, the nanoparticles (except for portions that contain binding ligands), may be provided with a passivating, non-reactive surface. For example, a coating of polyethylene glycol or another known surface passivating polymer may be applied to prevent protein interactions, nonspecific binding and aggregation, and so forth.

In accordance with another aspect of the invention, a kit is provided which contains at least first and second nanoparticle-containing injectable compositions. The first injectable composition comprises first self-assembling nanoparticles which comprise the following: (a) a first nanoparticle portion (b) tissue binding ligands attached to the first nanoparticle portion which result in the preferential binding and accumulation of the nanoparticles at one or more target locations in the body, and (c) first interparticle binding ligands attached to the first nanoparticle portion to promote interparticle binding. The second injectable composition comprises second self-assembling nanoparticles which comprise the following: (a) a second nanoparticle portion and (b) second interparticle binding ligands attached to the second nanoparticle portion, which preferentially bind to the first interparticle binding ligands attached to the first nanoparticle portion. The second self-assembling nanoparticles may or may not contain tissue binding ligands. Moreover, the first and second nanoparticle portions can be of the same or of different compositions.

In this aspect of the invention, injection of the first composition results in preferential binding and accumulation of the first self-assembling nanoparticles at one or more target locations in the body, thereby forming an initial base layer. Upon subsequent injection of the second composition, the interparticle binding ligands on the second nanoparticles preferentially bind to the first interparticle binding ligands of the first nanoparticles. By alternating the injection of the first and second compositions, nanoparticles are assembled on the tissue in a layer-by-layer fashion with lock-and-key specificity.

If desired, a third composition can then be administered which comprises third self-assembling nanoparticles which comprise the following: (a) a third nanoparticle portion and (b) third interparticle binding ligands attached to the third nanoparticle portion, which preferentially bind to the second interparticle binding ligands of second nanoparticles. Although tissue binding ligands can be attached to the third self-assembling nanoparticles, in many embodiments, the third self-assembling nanoparticles will not comprise tissue binding ligands. The nanoparticle portions of the third self-assembling nanoparticles can be the same as or different from the nanoparticle portions of the first and second self-assembling nanoparticles. Moreover, the third interparticle binding ligands can be the same as or different from the first interparticle binding ligands. Upon injection of the third composition, the interparticle binding ligands on the third self-assembling nanoparticles bind to those on the previously attached second self-assembling nanoparticles.

Analogous to the above, by alternating the injection of the second and third compositions, nanoparticles are assembled on the tissue in a layer-by-layer fashion.

As previously indicated, the compositions of the present invention can be used to in the treatment of a variety of diseases and conditions. “Treatment” refers to the prevention of a disease or condition, the reduction or elimination of symptoms associated with a disease or condition, or the substantial or complete elimination of a disease or condition. Preferred subjects (also referred to as “patients”) are vertebrate subjects, more preferably mammalian subjects and more preferably human subjects. For example, in various embodiments, the compositions of the present invention are used to form self-assembled structures at sites of atherosclerotic plaque, at aneurysmal sites, at myocardial infarcts, at infectious sites, at sites of vascular damage, and so forth.

As is typical for injectable compositions, the compositions of the present invention can include one or more pharmaceutically acceptable excipients or vehicles such as water, saline, glycerol, polyethylene-glycol, hyaluronic acid, ethanol, etc. Additionally, various auxiliary substances, such as wetting or emulsifying agents, biological buffering substances, and the like, may be present in such vehicles. A biological buffer can be virtually any solution which is pharmacologically acceptable and which provides the formulation with the desired pH, i.e., a pH in the physiological range. Examples of buffer solutions include saline, phosphate buffered saline, Tris buffered saline, Hank's buffered saline, and the like.

As noted above, the self-assembling nanoparticles within the compositions of the present invention have nanoparticle portions with attached ligands, including tissue binding and/or interparticle binding ligands, each of which will be discussed below.

The nanoparticle portions for use in the compositions of the present invention include organic nanoparticle portions (i.e., nanoparticle portions comprising at least 50 wt % organic molecules) such as polymeric nanoparticle portions (i.e., nanoparticle portions comprising at least 50 wt % polymer molecules), and inorganic nanoparticle portions (i.e., nanoparticle portions comprising at least 50 wt % inorganic molecules or atoms) such as metallic nanoparticle portions (i.e., nanoparticle portions comprising at least 50 wt % metal atoms) and non-metallic nanoparticle portions (i.e., nanoparticle portions comprising at least 50 wt % non-metallic atoms).

The nanoparticle portions of the present invention can have essentially any shape and include spheres, flat or bent plates, and linear or bent elongate particles which can be any cross section including circular, annular, polygonal, irregular, and so forth (e.g., elongated cylinders, tubes, columnar shapes with polygonal cross-sections, ribbon-shaped particles, etc.), as well as other regular or irregular geometries. The dimensions of the nanoparticles can vary widely, with largest dimensions (e.g., the diameter for a sphere, the width for a plate, the length for a rod, etc.) ranging anywhere from 1 to 1,000 nm, and smallest dimensions (e.g., the diameter of a rod, the thickness of a plate, etc.) ranging anywhere from 0.1 to 100 nm.

Polymers from which the nanoparticle portions can be formed include polymers which are natural and synthetic, biodegradable or non-biodegradable, homopolymeric or copolymeric, thermoplastic or non-thermoplastic, and so forth. Suitable polymers for forming the nanoparticle portions can be selected, for example, from the following: polycarboxylic acid polymers and copolymers including polyacrylic acids; acetal polymers and copolymers; acrylate and methacrylate polymers and copolymers (e.g., n-butyl methacrylate); cellulosic polymers and copolymers, including cellulose acetates, cellulose nitrates, cellulose propionates, cellulose acetate butyrates, cellophanes, rayons, rayon triacetates, and cellulose ethers such as carboxymethyl celluloses and hydroxyalkyl celluloses; polyoxymethylene polymers and copolymers; polyimide polymers and copolymers such as polyether block imides, polyamidimides, polyesterimides, and polyetherimides; polysulfone polymers and copolymers including polyarylsulfones and polyethersulfones; polyamide polymers and copolymers including nylon 6,6, nylon 12, polycaprolactams and polyacrylamides; resins including alkyd resins, phenolic resins, urea resins, melamine resins, epoxy resins, allyl resins and epoxide resins; polycarbonates; polyacrylonitriles; polyvinylpyrrolidones (cross-linked and otherwise); polymers and copolymers of vinyl monomers including polyvinyl alcohols, polyvinyl halides such as polyvinyl chlorides, ethylene-vinylacetate copolymers (EVA), polyvinylidene chlorides, polyvinyl ethers such as polyvinyl methyl ethers, polystyrenes, styrene-maleic anhydride copolymers, styrene-butadiene copolymers, styrene-ethylene-butylene copolymers (e.g., a polystyrene-polyethylene/butylene-polystyrene (SEBS) copolymer, available as Kraton® G series polymers), styrene-isoprene copolymers (e.g., polystyrene-polyisoprene-polystyrene), acrylonitrile-styrene copolymers, acrylonitrile-butadiene-styrene copolymers, styrene-butadiene copolymers and styrene-isobutylene copolymers (e.g., polyisobutylene-polystyrene block copolymers such as SIBS), polyvinyl ketones, polyvinylcarbazoles, and polyvinyl esters such as polyvinyl acetates; polybenzimidazoles; ionomers; polyalkyl oxide polymers and copolymers including polyethylene oxides (PEO); glycosaminoglycans; polyesters including polyethylene terephthalates and aliphatic polyesters such as polymers and copolymers of lactide (which includes lactic acid as well as d-,l- and meso lactide), epsilon-caprolactone, glycolide (including glycolic acid), hydroxybutyrate, hydroxyvalerate, para-dioxanone, trimethylene carbonate (and its alkyl derivatives), 1,4-dioxepan-2-one, 1,5-dioxepan-2-one, and 6,6-dimethyl-1,4-dioxan-2-one (a copolymer of polylactic acid and polycaprolactone is one specific example); polyether polymers and copolymers including polyarylethers such as polyphenylene ethers, polyether ketones, polyether ether ketones; polyphenylene sulfides; polyisocyanates; polyolefin polymers and copolymers, including polyalkylenes such as polypropylenes, polyethylenes (low and high density, low and high molecular weight), polybutylenes (such as polybut-1-ene and polyisobutylene), poly-4-methyl-pen-1-enes, ethylene-alpha-olefin copolymers, ethylene-methyl methacrylate copolymers and ethylene-vinyl acetate copolymers; polyolefin elastomers (e.g., santoprene), ethylene propylene diene monomer (EPDM) rubbers, fluorinated polymers and copolymers, including polytetrafluoroethylenes (PTFE), poly(tetrafluoroethylene-co-hexafluoropropene) (FEP), modified ethylene-tetrafluoroethylene copolymers (ETFE), and polyvinylidene fluorides (PVDF); silicone polymers and copolymers; polyurethanes; p-xylylene polymers; polyiminocarbonates; copoly(ether-esters) such as polyethylene oxide-polylactic acid copolymers; polyphosphazines; polyalkylene oxalates; polyoxaamides and polyoxaesters (including those containing amines and/or amido groups); polyorthoesters; biopolymers, such as polypeptides, proteins, polysaccharides and fatty acids (and esters thereof), including fibrin, fibrinogen, collagen, elastin, chitosan, gelatin, starch, glycosaminoglycans such as hyaluronic acid; as well as blends and further copolymers of the above.

Suitable metals from which nanoparticle portions can be formed can be selected include, for example, the following: substantially pure metals, such as silver, gold, platinum, palladium, iridium, osmium, rhodium, titanium, tungsten, and ruthenium, as well as metal alloys such as cobalt-chromium alloys, nickel-titanium alloys (e.g., nitinol), iron-chromium alloys (e.g., stainless steels, which contain at least 50% iron and at least 11.5% chromium), cobalt-chromium-iron alloys (e.g., elgiloy alloys), and nickel-chromium alloys (e.g., inconel alloys), among others.

Suitable non-metallic inorganic materials from which the nanoparticle portions can be formed can be selected include, for example, the following: calcium phosphate ceramics (e.g., hydroxyapatite); calcium-phosphate glasses, sometimes referred to as glass ceramics (e.g., bioglass); metal oxides, including non-transition metal oxides (e.g., oxides of metals from groups 13, 14 and 15 of the periodic table, including, for example, aluminum oxide) and transition metal oxides (e.g., oxides of metals from groups 3, 4, 5, 6, 7, 8, 9, 10, 11 and 12 of the periodic table, including, for example, oxides of titanium, zirconium, hafnium, tantalum, molybdenum, tungsten, rhenium, iridium, and so forth); carbon based materials such as pure and doped carbon (e.g., fullerenes, carbon nanofibers, single-wall, so-called “few-wall” and multi-wall carbon nanotubes), silicon carbides and carbon nitrides; silica (see Lu Y et al., “Aerosol-assisted self-assembly of mesostructured spherical nanoparticles,” Nature 398, 223-226, 18 Mar. 1999); synthetic or natural silicates including aluminum silicate, monomeric silicates such as polyhedral oligomeric silsequioxanes (POSS), including various functionalized POSS and polymerized POSS, and phyllosilicates including clays and micas (which may optionally be intercalated and/or exfoliated) such as montmorillonite, hectorite, hydrotalcite, vermiculite and laponite.

In some embodiments, nanoparticle portions are delivered in a physical configuration that differs from their ultimate physical configuration in vivo. For example, in some embodiments, the nanoparticle portions are formed using shape memory materials, such as shape memory metals or shape memory polymers. Shape-memory materials have the ability to memorize a shape. Exposure to a suitable stimulus, such as heat, causes a transition of the materials from a temporary shape to their memorized shape. For example, materials can be selected that go from a less compact configuration (e.g., a linear configuration, such as a straight or flat configuration) to a more compact configuration (e.g., a non-linear or non-planar configuration, such as a coiled or otherwise bent configuration), or vice versa.

For instance, nickel-titanium films can be deposited using techniques, such as vacuum thermal evaporation, electroplating or sputtering. For this purpose, a substrate is selected which may be, for example, completely etched or dissolved at a later point in the process (e.g., a substrate formed from silicon or from a salt such as NaCl, KCl, or NaF₂), or which may be formed of a material that is not ultimately etched (e.g., silicon with a polymer coating such as a polyimide film to produce a smooth, regular surface), but over which is provided a layer that is etched, for example, chromium or another material (e.g., aluminum or copper) having a highly specific etch rate relative to the nickel-titanium alloy so that the sacrificial layer may be removed without significantly etching the nickel-titanium alloy thin film. The sacrificial layer may be formed by conventional thin-film deposition techniques, such as vacuum thermal evaporation, electroplating or sputtering, to form a sacrificial layer preferably less than 1 micron in thickness. An etchant such as potassium hydroxide may be used for selectively etching aluminum, nitric acid may be used for selectively etching copper, and an etching solution containing ceric ammonium nitrate, nitric acid, and water (Chrome Etch from Arch Chemicals Inc.) may be used for selectively etching chromium. Nickel-titanium shape-memory alloy can then be sputter deposited, for example, using a sputter target composed of a nickel-titanium alloy (e.g., containing about 50 atomic percent titanium, 50 atomic percent nickel) The alloy composition may be enriched in nickel (e.g., by as much as 1-2 percent) to adjust the transition temperature as needed. The target is sputtered in a high-vacuum sputtering apparatus. When a desired film thickness is reached, the sputter deposition step is terminated. Further information on nickel-titanium film formation can be found in U.S. Patent Application No. 2003/0127318 to Johnson et al., which is hereby incorporated by reference in its entirety.

After sputtering is completed, distinct nickel-titanium alloy nanoparticles are formed on the substrate using metal masking and etching techniques such as those that are well known in the semiconductor industry. For example, a mask can be formed lithographically, followed by selective etching of certain areas of the nickel-titanium alloy through apertures in the mask (e.g., using a plasma etching process). Lithographic techniques have advanced rapidly. For example, the use of light coupling masks (LCM) for optical lithography has produced 80 nm features on a 200 nm pitch, using 248 nm illumination. Even smaller structures may be produced, for example, by resorting to extreme ultraviolet lithography, X-ray lithography and/or electron beam lithography. The mask can be removed after etching to expose the now discrete nanoparticles.

Subsequently, the film is annealed under heating/cooling conditions to achieve desired shape-memory alloy properties in the device. The annealing step may be, for example, by thermal heating or by exposure to an infrared heater in vacuum. Following annealing, the particles are released, for example, by exposure to a dissolving/etching solution as discussed above. For further information on annealing and film release, see U.S. Patent Application No. 2003/0127318.

When the nanoparticles are deformed and subsequently heated above the transition temperature, they revert to their original as-deposited shape, which may be example, a planar (i.e., flat) configuration or to a non-planar (e.g., bent) configuration, depending on the shape of the substrate on which they are deposited. For examples, in the former case, particles that have been bent at lower temperature will revert to a flattened configuration upon heating. Conversely, in the latter case, particles that are flattened at a lower temperature will bend upon heating. Nanoparticles may be bent or flatted at low temperature, for example, by depositing the film on a piezoelectric or electroactive polymer substrate and bent on demand. In addition, mechanical formation (e.g., pressing) is used on still other embodiments.

As an alternative to the above, a nickel-titanium alloy film having a graded composition can be formed, for example, as described in U.S. Patent Application No. 20030162048 to Ho et al. By gradual heating of the target during deposition of the thin film, a compositionally graded film is produced. Because the shape memory transition temperature in nickel-titanium alloy is very sensitive to composition, a bimorphic film of austenite and martensite is produced by this technique that exhibits a two-way shape memory effect without the need for further heat treatment. Hence, such films take on a first shape when heated, while reverting to a second shape when cooled. After forming the film, it is then patterned into nanoparticles and released as discussed above. Assuming that a flat substrate is used, the film curls when heated and returns to a flat configuration when cooled.

Another way of achieving a two-way shape memory effect is to introduce a biasing force by tailoring precipitates in the film such that there are compressive and tensile stresses on opposite sides of the film. See K. Kuribayashi, et al., “Micron sized arm using reversible TiNi alloy tin film actuators,” Mat. Res. Soc. Symp. Pro., vol. 276, p. 167, 1992. This film curls when in the martensitic phase and when heated to the austenite phase it is flattened because the higher modulus overcomes the residual stresses.

Other materials are available, besides metals, which exhibit a shape memory effect, including shape memory polymers. For example, U.S. Patent Application No. 2003/0055198 to Langer et al. describes various polymers having a shape memory effect.

Shape memory polymers frequently contain phase separated block co-polymers that have a hard segment and a soft segment. The melting point or glass transition temperature (Tₜᵣₐₙₛ) of the soft segment is substantially less than the melting point or glass transition temperature (Tₜᵣₐₙₛ) of the hard segment. When the shape memory polymer is heated above the Tₜᵣₐₙₛ of the hard segment, the material can be shaped. This first shape can be memorized by cooling the shape memory polymer below the Tₜᵣₐₙₛ of the hard segment. When the material is in a second shape at a temperature that is lower than the Tₜᵣₐₙₛ of the soft segment, the first shape is recovered by heating the material above the Tₜᵣₐₙₛ of the soft segment but below the Tₜᵣₐₙₛ of the hard segment. Examples of polymers used to prepare hard and soft segments of shape memory polymers vary widely and include various polyethers, polyacrylates, polyamides, polysiloxanes, polyurethanes, polyether amides, polyurethane/ureas, polyether esters, and urethane/butadiene copolymers.

U.S. Patent Application No. 2003/0055198 also describes a wide range of shape memory polymer compositions, which include a hard segment and at least one soft segment, and which can hold more than one shape in memory, if desired. At least one of the hard or soft segments can contain a crosslinkable group, and the segments can be linked by formation of an interpenetrating network or a semi-interpenetrating network, or by physical interactions of the blocks. Objects can be formed into a given shape at a temperature above the Tₜᵣₐₙₛ of the hard segment, and cooled to a temperature below the Tₜᵣₐₙₛ of the soft segment. If the object is subsequently formed into a second shape, the object can return to its original shape by heating the object above the Tₜᵣₐₙₛ of the soft segment and below the Tₜᵣₐₙₛ of the hard segment. The compositions can also include two soft segments which are linked via functional groups that are cleaved in response to application of light, electric field, magnetic field or ultrasound. The cleavage of these groups causes the object to return to its original shape. The hard and soft segments can be selected, for example, from polyhydroxy acids, polyorthoesters, polyether esters such as oligo(p-dioxanone), polyesters, polyamides, polyesteramides, polydepsidpetides, aliphatic polyurethanes, polysaccharides, polyhydroxyalkanoates, and copolymers thereof.

Once an appropriate polymer is selected, a layer of it is deposited of a substrate, for example, using thermoplastic or solvent casting techniques. As with metals, techniques for selectively masking and etching polymeric layers are well known in the semiconductor industry, where polymers are often employed, for example, due to their low dielectic constants. Once formed on the surface, the nanoparticles can be released by substrate/sacrificial layer etching as described above. As also described above, these nanoparticles can be processed to have a shape memory before being released, with the memorized shape depending on the shape of the substrate. Similar to the above, the nanoparticles are bent or flattened from their memorized shape on demand in some embodiments by depositing the polymer film on a piezoelectric or electroactive polymer or shape memory metal substrate. Moreover, residual stresses during formation may also be sufficient to bend or flatten the nanoparticles, thereby avoiding the need for actual deformation. In addition, mechanical formation (e.g., pressing) is used on still other embodiments.

In accordance with another specific embodiment of the invention, heat shrinkable nanoparticles are employed other than shape memory materials. For example, collagen particles having diameters ranging from about 3 to 40 microns, and with a minimum diameter of about 0.1 micron, have been prepared by emulsifying and cross-linking native collagen. Rossler B, et al., “Collagen microparticles: preparation and properties,” J. Microencapsul.; 1995 January-February; 12(1): 49-57. The particle size is primarily controlled by the molecular weight of the collagen that was used, with an increase in denaturation of the collagen resulting in smaller particle sizes. Id. It is well known that collagen shrinks when heated. Haines, B M, “Shrinkage temperature in collagen fibres.” Leather Conservation News, 3:1-5, 1987.

Magnetostrictive particles are also known, which change their size when a magnetic field is applied.

Hence, using the above and other techniques, nanoparticles can be formed which change shape when exposed to a suitable stimulus, such as heat. Consequently, once attached to tissues within the body, these nanoparticles can be activated to change shape.

For example, certain of these materials can be activated via localized application of heat or other stimulus (e.g., via a catheter or other insertable instrument).

Alternatively, certain of these materials can be activated using ex vivo stimulation to achieve an in vivo shape change. Sources of ex vivo stimulation include, for instance, oscillating magnetic fields, electromagnetic radiation (e.g., RF and microwave radiation), ultrasound, and so forth.

For example, magnetic nanoparticles can be heated by inductive heating using an oscillating magnetic field. To the extent that the material of interest is not intrinsically magnetic, magnetic nanoparticles, such as ferrite nanoparticles, can be added as susceptor particles. Alternatively, the material can be heated in situ using focused radiofrequency radiation, microwave radiation or ultrasound.

In some embodiments, the nanoparticles of the present invention are further provided with a drug, which can be delivered in vivo after self-assembly of nanoparticles.

As opposed to particles having only tissue binding ligands, the nanoparticles described herein contain interparticle binding ligands as well, allowing them to continue with interparticle assembly beyond the point of tissue contact. Consequently, self-assembled structures are formed in accordance with the present invention, which contain enhanced quantities of drugs.

“Drugs,” “therapeutic agents,” “pharmaceutically active agents,” and other related terms may be used interchangeably herein and include genetic and non-genetic Therapeutic agents may be used singly or in combination. Therapeutic agents may be, for example, nonionic or they may be anionic and/or cationic in nature.

Exemplary non-genetic therapeutic agents for use in connection with the present invention include: (a) anti-thrombotic agents such as heparin, heparin derivatives, urokinase, and PPack (dextrophenylalanine proline arginine chloromethylketone) and tissue plasminogen activator (TPA); (b) anti-inflammatory agents such as dexamethasone, prednisolone, corticosterone, budesonide, estrogen, sulfasalazine and mesalamine; (c) antineoplastic/antiproliferative/anti-miotic agents such as paclitaxel (including particulate forms thereof such as ABRAXANE albumin-bound paclitaxel nanoparticles), 5-fluorouracil, cisplatin, vinblastine, vincristine, epothilones, endostatin, angiostatin, angiopeptin, monoclonal antibodies capable of blocking smooth muscle cell proliferation, and thymidine kinase inhibitors; (d) anesthetic agents such as lidocaine, bupivacaine and ropivacaine; (e) anti-coagulants such as D-Phe-Pro-Arg chloromethyl ketone, an RGD peptide-containing compound, heparin, hirudin, antithrombin compounds, platelet receptor antagonists, anti-thrombin antibodies, anti-platelet receptor antibodies, aspirin, prostaglandin inhibitors, platelet inhibitors and tick antiplatelet peptides; (f) vascular cell growth promoters such as growth factors, transcriptional activators, and translational promotors; (g) vascular cell growth inhibitors such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin; (h) protein kinase and tyrosine kinase inhibitors (e.g., tyrphostins, genistein, quinoxalines); (i) prostacyclin analogs; (j) cholesterol-lowering agents; (k) angiopoietins; (l) antimicrobial agents such as triclosan, cephalosporins, aminoglycosides and nitrofurantoin; (m) cytotoxic agents, cytostatic agents and cell proliferation affectors; (n) vasodilating agents; (o) agents that interfere with endogenous vasoactive mechanisms; (p) inhibitors of leukocyte recruitment, such as monoclonal antibodies; (q) cytokines; (r) hormones; (s) inhibitors of HSP 90 protein (i.e., Heat Shock Protein, which is a molecular chaperone or housekeeping protein and is needed for the stability and function of other client proteins/signal transduction proteins responsible for growth and survival of cells) including geldanamycin; (t) beta-blockers, (u) bARKct inhibitors, (v) phospholamban inhibitors, and (w) Serca 2 gene/protein.

Exemplary genetic therapeutic agents for use in connection with the present invention include anti-sense DNA and RNA as well as DNA coding for the various proteins (as well as the proteins themselves): (a) anti-sense RNA, (b) tRNA or rRNA to replace defective or deficient endogenous molecules, (c) angiogenic and other factors including growth factors such as acidic and basic fibroblast growth factors, vascular endothelial growth factor, endothelial mitogenic growth factors, epidermal growth factor, transforming growth factor α and β, platelet-derived endothelial growth factor, platelet-derived growth factor, tumor necrosis factor α, hepatocyte growth factor and insulin-like growth factor, (d) cell cycle inhibitors including CD inhibitors, and (e) thymidine kinase (“TK”) and other agents useful for interfering with cell proliferation. Also of interest is DNA encoding for the family of bone morphogenic proteins (“BMP's”), including BMP-2, BMP-3, BMP-4, BMP-5, BMP-6 (Vgr-1), BMP-7 (OP-1), BMP-8, BMP-9, BMP-10, BMP-11, BMP-12, BMP-13, BMP-14, BMP-15, and BMP-16. Currently preferred BMP's are any of BMP-2, BMP-3, BMP-4, BMP-5, BMP-6 and BMP-7. These dimeric proteins can be provided as homodimers, heterodimers, or combinations thereof, alone or together with other molecules. Alternatively, or in addition, molecules capable of inducing an upstream or downstream effect of a BMP can be provided. Such molecules include any of the “hedgehog” proteins, or the DNA's encoding them.

Vectors for delivery of genetic therapeutic agents include viral vectors such as adenoviruses, gutted adenoviruses, adeno-associated virus, retroviruses, alpha virus (Semliki Forest, Sindbis, etc.), lentiviruses, herpes simplex virus, replication competent viruses (e.g., ONYX-015) and hybrid vectors; and non-viral vectors such as artificial chromosomes and mini-chromosomes, plasmid DNA vectors (e.g., pCOR), cationic polymers (e.g., polyethyleneimine, polyethyleneimine (PEI)), graft copolymers (e.g., polyether-PEI and polyethylene oxide-PEI), neutral polymers such as polyvinylpyrrolidone (PVP), SP1017 (SUPRATEK), lipids such as cationic lipids, liposomes, lipoplexes, nanoparticles, or microparticles, with and without targeting sequences such as the protein transduction domain (PTD).

Numerous therapeutic agents, not necessarily exclusive of those listed above, have been identified as candidates for vascular treatment regimens, for example, as agents targeting restenosis. Such agents are useful for the practice of the present invention and include one or more of the following: (a) Ca-channel blockers including benzothiazapines such as diltiazem and clentiazem, dihydropyridines such as nifedipine, amlodipine and nicardapine, and phenylalkylamines such as verapamil, (b) serotonin pathway modulators including: 5-HT antagonists such as ketanserin and naftidrofuryl, as well as 5-HT uptake inhibitors such as fluoxetine, (c) cyclic nucleotide pathway agents including phosphodiesterase inhibitors such as cilostazole and dipyridamole, adenylate/guanylate cyclase stimulants such as forskolin, as well as adenosine analogs, (d) catecholamine modulators including α-antagonists such as prazosin and bunazosine, β-antagonists such as propranolol and α/β-antagonists such as labetalol and carvedilol, (e) endothelin receptor antagonists, (f) nitric oxide donors/releasing molecules including organic nitrates/nitrites such as nitroglycerin, isosorbide dinitrate and amyl nitrite, inorganic nitroso compounds such as sodium nitroprusside, sydnonimines such as molsidomine and linsidomine, nonoates such as diazenium diolates and NO adducts of alkanediamines, S-nitroso compounds including low molecular weight compounds (e.g., S-nitroso derivatives of captopril, glutathione and N-acetyl penicillamine) and high molecular weight compounds (e.g., S-nitroso derivatives of proteins, peptides, oligosaccharides, polysaccharides, synthetic polymers/oligomers and natural polymers/oligomers), as well as C-nitroso-compounds, O-nitroso-compounds, N-nitroso-compounds and L-arginine, (g) Angiotensin Converting Enzyme (ACE) inhibitors such as cilazapril, fosinopril and enalapril, (h) ATII-receptor antagonists such as saralasin and losartin, (i) platelet adhesion inhibitors such as albumin and polyethylene oxide, (j) platelet aggregation inhibitors including cilostazole, aspirin and thienopyridine (ticlopidine, clopidogrel) and GP IIb/IIIa inhibitors such as abciximab, epitifibatide and tirofiban, (k) coagulation pathway modulators including heparinoids such as heparin, low molecular weight heparin, dextran sulfate and β-cyclodextrin tetradecasulfate, thrombin inhibitors such as hirudin, hirulog, PPACK(D-phe-L-propyl-L-arg-chloromethylketone) and argatroban, FXa inhibitors such as antistatin and TAP (tick anticoagulant peptide), Vitamin K inhibitors such as warfarin, as well as activated protein C, (l) cyclooxygenase pathway inhibitors such as aspirin, ibuprofen, flurbiprofen, indomethacin and sulfinpyrazone, (m) natural and synthetic corticosteroids such as dexamethasone, prednisolone, methprednisolone and hydrocortisone, (n) lipoxygenase pathway inhibitors such as nordihydroguairetic acid and caffeic acid, (o) leukotriene receptor antagonists, (p) antagonists of E- and P-selectins, (q) inhibitors of VCAM-1 and ICAM-1 interactions, (r) prostaglandins and analogs thereof including prostaglandins such as PGE1 and PGI2 and prostacyclin analogs such as ciprostene, epoprostenol, carbacyclin, iloprost and beraprost, (s) macrophage activation preventers including bisphosphonates, (t) HMG-CoA reductase inhibitors such as lovastatin, pravastatin, fluvastatin, simvastatin and cerivastatin, (u) fish oils and omega-3-fatty acids, (v) free-radical scavengers/antioxidants such as probucol, vitamins C and E, ebselen, trans-retinoic acid and SOD mimics, (w) agents affecting various growth factors including FGF pathway agents such as bFGF antibodies and chimeric fusion proteins, PDGF receptor antagonists such as trapidil, IGF pathway agents including somatostatin analogs such as angiopeptin and ocreotide, TGF-β pathway agents such as polyanionic agents (heparin, fucoidin), decorin, and TGF-β antibodies, EGF pathway agents such as EGF antibodies, receptor antagonists and chimeric fusion proteins, TNF-α pathway agents such as thalidomide and analogs thereof, Thromboxane A2 (TXA2) pathway modulators such as sulotroban, vapiprost, dazoxiben and ridogrel, as well as protein tyrosine kinase inhibitors such as tyrphostin, genistein and quinoxaline derivatives, (x) MMP pathway inhibitors such as marimastat, ilomastat and metastat, (y) cell motility inhibitors such as cytochalasin B, (z) antiproliferative/antineoplastic agents including antimetabolites such as purine analogs (e.g., 6-mercaptopurine or cladribine, which is a chlorinated purine nucleoside analog), pyrimidine analogs (e.g., cytarabine and 5-fluorouracil) and methotrexate, nitrogen mustards, alkyl sulfonates, ethylenimines, antibiotics (e.g., daunorubicin, doxorubicin), nitrosoureas, cisplatin, agents affecting microtubule dynamics (e.g., vinblastine, vincristine, colchicine, Epo D, paclitaxel and epothilone), caspase activators, proteasome inhibitors, angiogenesis inhibitors (e.g., endostatin, angiostatin and squalamine), rapamycin, cerivastatin, flavopiridol and suramin, (aa) matrix deposition/organization pathway inhibitors such as halofuginone or other quinazolinone derivatives and tranilast, (bb) endothelialization facilitators such as VEGF and RGD peptide, and (cc) blood rheology modulators such as pentoxifylline.

Numerous additional therapeutic agents useful for the practice of the present invention are also disclosed in U.S. Pat. No. 5,733,925 assigned to NeoRx Corporation, the entire disclosure of which is incorporated by reference.

In some embodiments, drugs are linked to the nanoparticle portions using covalent coupling techniques such as those discussed below in conjunction with ligand coupling.

In some embodiments, drugs are provided within nanocapsules, which either correspond to the nanoparticle portion or which are coupled to the nanoparticle portion. In this connection, polyelectrolyte nanocapsules have a number of desirable properties that make them useful for purposes of the present invention. For example, they permit the encapsulation of a wide variety of therapeutic and other agents, including small molecule pharmaceuticals, polypeptides (e.g., proteins such as enzymes), polynucleotides (e.g., DNA and RNA), and so forth. See, e.g., “Microencapsulation of Organic Solvents in Polyelectrolyte Multilayer Micrometer-sized Shells,” S. Moya et al., Journal of Colloid and Interface Science, 216, 297-302 (1999). In addition, drugs can be loaded within these nanocapsules with high precision, for example, in multiples of 0.1 pico-gram per nanocapsule. See, e.g., “Assembly of Alternated Multivalent Ion/Polyelectrolyte Layers on Colloidal Particles,” I. L. Radtchenko et al., Journal of Colloid and Interface Science, 230, 272-280 (2000).

Polyelectrolyte nanocapsules can be prepared using various known layer-by-layer techniques. Layer-by-layer techniques typically involve coating particles or droplets dispersed in aqueous media via electrostatic, self-assembly using charged polymeric (polyelectrolyte) materials. These techniques exploit the fact that the particles or droplets serving as templates for the polyelectrolyte layers each has a surface charge. This renders the particles water dispersible and provides the charge necessary for deposition of subsequent polyelectrolyte layers. Multilayers are formed by repeated treatment with alternating oppositely charged polyelectrolytes, i.e., by alternating treatment with cationic and anionic polyelectrolytes. The polymer layers self-assemble onto the pre-charged solid/liquid particles by means of electrostatic, layer-by-layer deposition, thus forming a multilayered polymeric shell around the cores.

Many materials, such as polypeptides and polynucleotides, have an inherent surface charge that is present on particles made from the same. Other materials are uncharged. Such materials, however, can nonetheless be encapsulated by layer-by-layer techniques, for example, by providing them within particles or droplets which have a surface charge

Polyelectrolytes are polymers having ionically dissociable groups, which can be a component or substituent of the polymer chain. Usually, the number of these ionically dissociable groups in the polyelectrolytes is so large that the polymers in dissociated form (also called polyions) are water-soluble. Specific examples of polycations include protamine sulfate polycations, poly(allylamine) polycations (e.g., poly(allylamine hydrochloride) (PAH)), polydiallyldimethylammonium polycations, polyethyleneimine polycations, chitosan polycations, eudragit polycations, gelatine polycations, spermidine polycations and albumin polycations. Specific examples of polyanions include poly(styrenesulfonate) polyanions (e.g., poly(sodium styrenesulfonate) (PSS)), polyacrylic acid polyanions, sodium alginate polyanions, eudragit polyanions, gelatine polyanions, hyaluronic acid polyanions, carrageenan polyanions, chondroitin sulfate polyanions, and carboxymethylcellulose polyanions.

By using polyelectrolytes that are degradable, the release of enclosed drug can be controlled via the degradation of the nanocapsule walls. Otherwise, release is typically controlled by diffusion.

The wall thickness provided by layer-by-layer techniques will frequently range, for example, from 4 to 50 nm. The size of the resulting nanocapsules can vary widely, depending upon the size of the template, and will frequently range, for example, from 50 to 1000 nanometers in largest dimension, but dimensions beyond these values may also be provided.

Techniques other than direct encapsulation are also available for encapsulating agents of interest within polyelectrolyte shells. For example, various techniques take advantage of gradients across the nanocapsule wall to effect precipitation or synthesis of a desired substance within the shell. For instance, as a general rule, large macromolecules typically cannot penetrate polyelectrolyte multilayers, while small molecules, on the other hand, can. Accordingly, the presence of macromolecules inside the nanocapsules can lead to a difference in the physico-chemical properties between the inside and the outside of the nanocapsule, for example, providing gradients in pH and/or polarity that can be used to trap materials within the nanocapsules.

Moreover, charged drugs can be substituted for one or more of the polyelectrolyte layers, thereby incorporating the drug between polyelectrolyte layers within the capsule shell.

Materials instead of, or addition to, drugs can also be encapsulated using polyelectrolyte deposition techniques. As a specific example, the encapsulation of magnetite (Fe₃O₄) nanoparticles inside poly(styrene sulfonate)/poly(allylamine hydrochloride polyelectrolyte multilayers has been reported. Micron and submicron sized nanocapsules are made by means of layer-by-layer adsorption of oppositely charged polyelectrolytes (PSS, PAH) on the surface of colloidal template particles (e.g., weakly cross-linked melamine formaldehyde particles having a precipitated PAH-citrate complex) with subsequent degradation of the template core. This leaves free PAH in the core, which creates a pH gradient across the shell. At this point, (a) negatively charged, preformed magnetic particles of sufficiently small size (e.g., Fe₃O₄ nanoparticles) can be used to impregnate the nanocapsules whereupon they are held by electrostatic interactions, or (b) magnetic material (e.g., Fe₃O₄) can be selectively synthesized inside the core based on the pH gradient and on presence of dissolved PAH in the nanocapsule. The resulting nanocapsules are easily driven by a magnetic field. Additional information can be found, for example, in “Micron-Scale Hollow Polyelectrolyte Nanocapsules with Nanosized Magnetic Fe₃O₄ Inside,” Materials Letters, D. G. Shchukin et al. (in press), the disclosure of which is hereby incorporated by reference. If desired, drugs can be incorporated into such nanocapsules along with the magnetic material.

Further information on the formation of nanocapsules having polyelectrolyte shells can be found, for example, in U.S. patent application Ser. No. 10/638,739, United States Patent Application Pub. No. 2002/0187197, WO 99/47252, WO 00/03797, WO 00/77281, WO 01/51196, WO 02/09864, WO 02/09865, WO 02/17888, “Fabrication of Micro Reaction Cages with Tailored Properties,” L. Dähne et al., J. Am. Chem. Soc., 123, 5431-5436 (2001), “Lipid Coating on Polyelectrolyte Surface Modified Colloidal Particles and Polyelectrolyte Nanocapsules,” Moya et al., Macromolecules, 33, 4538-4544 (2000); “Controlled Precipitation of Dyes into Hollow Polyelectrolyte Nanocapsules,” G. Sukhorukov et al., Advanced Materials, Vol. 12, No. 2, 112-115 (2000), “A Novel Method for Encapsulation of Poorly Water-soluble Drugs: Precipitation in Polyelectrolyte Multilayer Shells,” I. L. Radtchenko et al., International Journal of pharmaceutics, 242, 219-223 (2002), the disclosures of which are hereby incorporated by reference.

For nanoparticles containing encapsulated drugs, drug release can occur, for example, due to one or more of the following mechanisms: (a) as a result of diffusion through the encapsulation layer or layers, (b) as a result of biodegradation of the encapsulation layer(s), and (c) as a result of increased permeability or breakage of the encapsulation layer(s), for example, due to external stimulation using radiofrequency radiation, microwave radiation, oscillating magnetic fields, or ultrasound (which can assist with delivery, for example, via the generation of thermal energy or via acoustic cavitation). For example, using Magnetic Resonance Imaging (MRI) fields at diagnostic levels, researchers at the Biological Systems Office (BSO), Johnson Space Center have heated microcapsules containing ferromagnetic particles to a temperature that is sufficient to melt holes in the outer skin of the microcapsules. Similarly, ferromagnetic nanoparticles within polyelectrolyte capsules (see above) could be likewise heated to the point where they penetrate the polyelectrolyte shell, so long shell materials are chosen which have melting temperatures that are below the temperatures attained by the ferromagnetic nanoparticles during heating.

For nanoparticles having attached drugs, release can occur, for example, due to one or more of the following mechanisms: (a) as a result of biodegradation of the nanoparticles, (b) as a result of biodegradation of a coupling species between the nanoparticles and the drugs, (c) by selection of a thermosensitive coupling, which is severed by heating the particle to which it is attached or the environment that it occupies (e.g., by exposure to ultrasound, alternating magnetic fields and radio- and microwave-frequency electromagnetic fields).

Some embodiments of the invention involve nanoparticles which can be heated in vivo to produce localized cell death, for example, by exposing the assembled nanoparticles to ultrasound, alternating magnetic fields and radio- and microwave-frequency electromagnetic fields as discussed above. Mechanisms of cell death due to heating include necrotic processes and apoptotic process. Necrotic cells undergo swelling and rupture, while apoptotic cells are removed by phagocytosis because they display markers on their cell surfaces that target them for selective elimination. Mild hyperthermia (e.g., 43° C. for 30 to 60 minutes) is known to enhance apoptosis in normal and cancerous cell populations, while higher temperatures (e.g., higher than 56° C.) trigger the necrotic process. For more information see, e.g., Andrea Jordan et al., “Presentation of a new magnetic field therapy system for the treatment of human solid tumors with magnetic fluid hyperthermia,” Journal of Magnetism and Magnetic Materials, 225 (2001) 118-126; and Kuznetsov A A et al., “‘Smart’ mediators for self-controlled inductive heating,” European Cells and Materials, Vol. 3. Suppl. 2, 2002 (pages 75-77).

As previously noted, an important feature of the present invention is that the nanoparticles self assemble in vivo. Self assembly is directed in the present invention by providing the nanoparticles with ligands, which attach to tissue in the body or which attach to ligands on other nanoparticles.

The ligands for binding the nanoparticles of the present invention to tissue will depend upon the tissue being targeted. Tissue attachment ligands can be selected, for example, the following species (or portions thereof): ankyrins, cadherins, members of the immunoglobulin superfamily (which includes a wide array of molecules, including NCAMs, ICAMs, VCAMs, and so forth), selectins (L-, E- and P-subclasses), proteoglycans, connexins, mucoadhesives, sialyl Lex, plant or bacterial lectins (adhesion molecules which specifically bind to sugar moieties of the epithelial cell membrane), laminins, dermatan sulphate, entactin, fibrin, fibronectin, vimentin, collagen, glycolipids, glycophorin, glycoproteins, heparan sulphate, heparin sulphate, hyaluronic acid, keratan sulphate, spektrin, von Willebrand factor, vinculin, vitronectin, and polypeptides and proteins containing various peptide sequences including RGD tripeptide (i.e., ArgGlyAsp, which has been identified to be responsible for some of the cell adhesion properties of fibronectin, laminin, collagen I, collagen IV, thrombospondin, and tenascin), REDV tetrapeptide (i.e., Arg-Glu-Asp-Val), which has been shown to support endothelial cell adhesion but not that of smooth muscle cells, fibroblasts, or platelets), and YIGSR pentapeptide (i.e., TyrIleGlySerArg, which promotes epithelial cell attachment, but not platelet adhesion). More information on these and other peptides can be found in U.S. Pat. No. 6,156,572 and U.S. Patent Application No. 2003/0087111.

In this connection, small oligopeptides (e.g., two to 12 amino acids) are normally rapidly removed from the body, with various processes involved in their clearance. See Meijer D K, et al., “Disease-induced drug targeting using novel peptide-ligand albumins,” J Control Release; 2001 May 14; 72(1-3):157-64. However, by coupling of such peptides to nanoparticles, elimination via various pathways is expected to be reduced or prevented.

Thus, interactions between ligands and tissues are selective in the present invention, with beneficial tissue-ligand interactions including ligand-cell receptor interactions, antibody-antigen type interactions (e.g., using whole antibodies or antibody fragments), interactions between enzymes and coenzymes and inhibitors, and nucleic acid hybridization, among other interactions.

A few specific examples of tissue targeting ligands are discussed in detail below.

For example, histologic features of vulnerable plaques include a large lipid core, a thin fibrous cap, intraplaque hemorrhage, and an increased number of inflammatory cells, particular monocyte-macrophages. Plaque is composed of a core (containing, for example, lipid and cholesterol crystals, macrophages, foam cells, necrotic cell debris, plasma proteins and degenerating blood elements) that is separated from the lumen by a layer of fibrous tissue, also known as a fibrous cap (containing, for example, smooth muscle cells, macrophages, foam cells, collagen, elastin, proteoglycans and other extracellular matrix [ECM] components).

Hence, where the targeted tissue is atherosclerotic plaque, ligands can be selected based on the presence or expression of various molecular species in the ECM components of the plaque. In particular, plaque remodelling is known to occur by matrix metalloproteinases (MMPs), specifically MMP-1, MMP-2, MMP-3 and MMP-9. See Zaltsman A B et al., “Increased secretion of tissue inhibitors of metalloproteinases 1 and 2 from the aortas of cholesterol fed rabbits partially counterbalances increased metalloproteinase activity,” Arterioscler Thromb Vasc Biol; 1999 July; 19(7):1700-7. Moreover, various tissue inhibitors of metalloproteinases (TIMPs) at known to preferentially bind to matrix metalloproteinases. For instance, TIMP-1 preferentially binds to MMP-1 and MMP-9, TIMP-2 preferentially binds to MMP-2, and TIMP-3 preferentially binds to MMP-1 and MMP-9. Id. Mutants of TIMPs have also been reported which have enhanced binding affinity to MMPs, including MMP-2 and MMP-3. Shuo Wei et al., “Protein Engineering of the Tissue Inhibitor of Metalloproteinase 1 (TIMP-1) Inhibitory Domain,” J. Biol. Chem., Vol. 278, Issue 11, 9831-9834, Mar. 14, 2003. Accordingly, in one embodiment of the invention, TIMPs, or analogs or derivatives thereof, are used for targeting plaque.

Antibodies are also available, or they can be generated using known techniques, for targeting MMPs in the fibrous cap. For example, rabbit anti-MMP-1 (which binds to MMP-1 but does not cross react with MMP family members MMP-2A, MMP-2B, and MMP-3, MMP-9), is available from Research Diagnostics Inc., Flandersi N.J., USA. Also available form Research Diagnostics Inc. are mouse anti-human MMP-3 monoclonal antibody, rabbit Anti-MMP-3 antibody, mouse anti-human MMP-9 monoclonal antibody, and rabbit anti-MMP-9 antibody. Accordingly, in another embodiment of the invention, anti-MMP antibodies, or fragments, analogs or derivatives thereof, are used for targeting plaque.

Type III collagen in the fibrous cap is another target for self-assembly, based on its exposure and the loss of the basement membrane that overlays the cap. See, e.g., Kolodgie F D et al., “Differential accumulation of proteoglycans and hyaluronan in culprit lesions: insights into plaque erosion,” Arterioscler Thromb Vasc Biol; 2002 Oct. 1; 22(10):1642-8. Antibodies are also available, or can be generated, for use in forming ligands that bind to collagen III. For example, mouse collagen type III monoclonal antibody, is available from Chemicon International, Inc, Temecula, Calif., USA and rabbit collagen III antibody and mouse collagen III antibody are available from Abcam, Ltd., Cambridge, UK. Hence, in another embodiment of the invention, anti collagen type III antibodies, or fragments, analogs or derivatives thereof, are used for targeting plaque.

Another target for self-assembly is lipoprotein (a) matrix metalloproteinase-derived F2, since this is present in regions of increased matrix metalloproteinase 2 and matrix metalloproteinase 9. See Fortunato J E et al., “Apolipoprotein (a) fragments in relation to human carotid plaque instability,” J Vasc Surg; 2000 September; 32(3):555-63.

Apoptosis is common in advanced human atheroma and contributes to plaque instability. Annexin V (a member of the annexin family of calcium-dependent phospholipid-binding proteins) has a high affinity for exposed phosphatidylserine on apoptotic cells. See Kolodgie F D, et al., “Targeting of apoptotic macrophages and experimental atheroma with radiolabeled annexin V: a technique with potential for noninvasive imaging of vulnerable plaque,” Circulation. 2003 Dec. 23; 108(25):3134-9. In addition, benzyloxycarbonyl-Val-Ala-DL-Asp(O-methyl)-fluoromethyl ketone (Z-VAD-fmk), is known to be a potent inhibitor of the enzymatic cascade intimately associated with apoptosis. See Haberkorn U, et al., “Investigation of a potential scintigraphic marker of apoptosis: radioiodinated Z-Val-Ala-DL-Asp(O-methyl)-fluoromethyl ketone,” Nucl Med Biol. 2001 October; 28(7):793-8.

Moreover, the presence of an inflammatory stimulus increases the expression of CC (cysteine-cysteine motif) chemokine receptor (CCR)-2 on monocytes and macrophages, as well as somatostatin receptors on T lymphocytes. Monocyte chemoattractant protein (MCP)-1 binds with high affinity to CCR-2 and is thus used to detect subacute and chronic inflammatory lesions. See Blankenberg F G, et al., “Development of radiocontrast agents for vascular imaging: progress to date,” Am J Cardiovasc Drugs; 2002; 2(6):357-65. In addition, octreotide or depreotide are used to detect activated T lymphocytes which may identify vulnerable plaque. Id. MCP-1 and fluoro-2-deoxyglucose have been shown in animal models to be effective in identifying macrophage infiltration and metabolic activity in atheromatous lesions, respectively. Id.

Furthermore, MDC, fractalkine, and TARC, which are chromosome 16q13 chemokines, are expressed in atherosclerotic lesions. Greaves D R et al., “Linked chromosome 16q13 chemokines, macrophage-derived chemokine, fractalkine, and thymus- and activation-regulated chemokine, are expressed in human atherosclerotic lesions,” Arterioscler Thromb Vasc Biol; 2001 June; 21(6):923-9.

Peptides such as endothelin are also being explored as agents for collecting at unstable atherosclerotic plaque. Knight L C, “Non-oncologic applications of radiolabeled peptides in nuclear medicine,” Q J Nucl Med; 2003 December; 47(4):279-91.

Consequently ligands containing annexin V, Z-VAD-fmk, (MCP)-1, octreotide, depreotide, fluoro-2-deoxyglucose, MDC, fractalkine, TARC and endothelin, among others, or fragments, analogs or derivatives thereof, are used in certain embodiments of the invention for targeting plaque.

In addition to plaque, apoptosis is also associated with cancer, acute cerebral and myocardial ischemic injury and infarction, immune mediated inflammatory disease and transplant rejection. See Blankenberg F G, “Recent advances in the imaging of programmed cell death,”, Curr Pharm Des, 2004; 10(13):1457-67 and Blankenberg F, et al., “Imaging cell death in vivo,” Q J Nucl Med; 2003 December; 47(4):337-48. Hence, in some embodiments of the invention, ligands containing species with a high affinity for apoptotic cells, such as annexin V and Z-VAD-fmk (or fragments, analogs or derivatives thereof), among others, are used for the treatment and/or diagnosis of these conditions as well.

With respect to infarcts, antimyosin Fab, a Fab monoclonal antibody fragment, is known to provide great specificity for the detection of myocardial necrosis, irrespective of the cause of injury. Khaw B A, “The current role of infarct avid imaging,” Semin Nucl Med; 1999 July; 29(3):259-70. For example, five patients with a history of remote infarction and acute necrosis were reported to show antimyosin uptake only in regions concordant with the acute episodes of infarction, and radiolabeled antimyosin Fab localized in neither old infarcts nor normal, noninfarcted myocardium. Khaw B A et al., “Acute myocardial infarct imaging with indium-111-labeled monoclonal antimyosin Fab,” J Nucl Med; 1987 November; 28(11):1671-8. Consequently ligands containing antimyosin Fab, or fragments, analogs or derivatives thereof, are used in some embodiments of the invention for targeting infarcts.

Autologous leukocytes concentrate at inflammatory and infectious sites, as do cytokines (e.g., IL-1, IL-2) and chemokines (e.g., IL-8, PF-4, MCP-1, NAP-2), complement factors (e.g., C5a and C5adR), chemotactic peptides (e.g., fMLF), other chemotactic factors (e.g., LTB4), as well as antagonists to the tuftsin receptor. van Eerd J E, et al., “Radiolabeled chemotactic cytokines: new agents for scintigraphic imaging of infection and inflammation,” Q J Nucl Med; 2003 December; 47(4):246-55 and Knight L C, “Non-oncologic applications of radiolabeled peptides in nuclear medicine,” Q J Nucl Med; 2003 December; 47(4):279-91. Hence, ligands containing these species, or fragments, analogs or derivatives thereof, are used in some embodiments of the invention for targeting inflammatory and infectious sites.

Expression of alpha(v)beta(3) integrin is increased in activated endothelial cells and vascular smooth muscle cells after vascular injury, whereas alpha(v)beta(3) integrin is minimally expressed on smooth muscle cells and is not expressed on quiescent epithelial cells. See Blankenberg F G, et al., “Development of radiocontrast agents for vascular imaging: progress to date,” Am J Cardiovasc Drugs; 2002; 2(6):357-65. Moreover, it is reported that radiolabeled high-affinity peptides can be used to target the alpha(v)beta(3) integrin and visualize areas of vascular damage. Id. Hence, ligands containing this peptide or fragments, analogs or derivatives thereof, are used in accordance with some embodiments of the invention for targeting vascular damage.

In vascular damage, sub-endothelial regions are exposed, such as the basal lamina/basement membrane (which is a network of specialized ECM proteins, including type IV collagen, fibronectin, laminin, heparan sulfate proteoglycan, and nidogen which is a sulphated glycoprotein), and for larger vessels, there is a tunica media (which is composed of smooth muscle cells within a matrix of elastin, type I, III and V collagen, proteoglycan, and so forth). Hence, ligands for targeting vascular damage also include various integrins which bind to theses species. Integrins recognize a wide variety of extracellular matrix components and cell-surface receptors, including collagen, fibronectin, vitronectin, laminin, fibrinogen, and adhesion molecules including intracellular adhesion molecules (ICAMS) and vascular adhesion molecules (VCAMS). Members of the integrin family of cell-surface receptors are expressed on virtually all mammalian cells and mediate adhesion of cells to one another and to the extracellular matrix. Additional information can be found, for example, in U.S. Patent Appln. No. 2002/0058336 and U.S. Pat. Appln. No. 2003/0007969, the disclosures of which are hereby incorporated by reference.

For thromboembolic disease, peptides which bind to various components of thrombi are known, including peptide analogs of fibrin or fragments of fibronectin which have a distinct binding domain for fibrin, linear and cyclic peptide antagonists of the glycoprotein IIb/IIIa receptor on platelets, naturally occurring antagonists of this receptor which are found in venoms, analogues of laminin and thrombospondin which bind to other receptors on platelets, and peptides which target thrombin that which is sequestered within a fibrin clot. Knight L C, “Non-oncologic applications of radiolabeled peptides in nuclear medicine,” Q J Nucl Med; 2003 December; 47(4):279-91. In certain of these embodiments, the nanoparticles are provided with agents to help resolve and heal the thrombus, such as plasmin, Tissue Plasminogen Activator (TPA), growth factors and/or cell adhesion proteins, such as fibronectin, RGD peptides, etc.

Nanoparticles in accordance with the present invention are also provided with ligands for interparticle binding. As with ligands for tissue binding, interactions between the interparticle binding ligands are selective and include such beneficial interactions as ligand-receptor type interactions, antibody-antigen type interactions, nucleic acid interactions, and cell receptive mimetic binding, among others. A specific example of an interparticle ligand binding pair is the combination of a synthetic peptide sequence (preferable having no in vivo counterpart) and an antibody (or antibody fragment) for the same.

Once a ligand is selected, it must be associated with a nanoparticle portion, for example, those described above. In this regard, recent years have seen an enormous increase in the development of techniques for coupling polypeptides, polysaccharides, polynucleotides, and other biopolymers as well and small molecules to solid supports. For example, coupling techniques are widely practiced for use in diagnostic applications, for instance, affinity chromatography.

In general, the immobilization technique selected will depend upon the chemical characteristics of the ligand (e.g., whether it is a polypeptide, polysaccharide, polynucleotide, small molecule substance, etc.) and the nanoparticle (e.g., whether it is organic or inorganic, metallic or non-metallic). Obviously, the technique should not destroy the binding ability of the ligand.

Depending on the available reactive groups within the selected ligand, several well known coupling chemistries are readily available for ligand immobilization, including those based on various condensation, addition, and substitution reactions. For example, amine, thiol and aldehyde coupling chemistries are well known in the coupling art. Most macromolecules contain amine groups, which can be used in amine coupling. The choice of thiol coupling depends on the availability of thiol groups on the ligand. However, it is relatively easy to provide a given ligand with thiol groups, if necessary. Thiol chemistry generally considered to be more robust than the amine chemistry, so the coupling conditions are less critical. The choice of aldehyde coupling is made, for instance, with polysaccharides and glycoconjugates. With respect to streptavidin-biotin coupling, nucleic acids, polysaccharides and glycoconjugates are relatively easy biotinylated using a variety of reagents and functional groups.

With respect to the nanoparticle portions, those that are polymeric in nature frequently have organic functional groups, which can directly participate, or can be readily modified to participate, in coupling chemistries known in the art for attaching ligands, including those discussed above.

Where the nanoparticles are metallic or ceramic in nature, the surfaces are typically derivatized prior to coupling. For example, using techniques such as those described in U.S. Ser. No. 10/830,772, ligands may be covalently coupled to a nanoparticle surface by a method that comprises: (a) halogenating the surface; and (b) reacting the halogenated surface with a reactive molecule that is covalently reactive with the chlorinated surface region. For example, the surface region may be halogenated by exposing the exposing the surface region to a reactive chloride, for example, a reactive chloride selected from the following: SiCl₄ (silicon tetrachloride), TiCl₄ (titanium tetrachloride), GeCl₄ (germanium tetrachloride), SnCl₄ (tin tetrachloride), VCl₄ (vanadium tetrachloride), MoCl₅ (molybdenum pentachloride), WCl₆ (tungsten hexachloride), BCl₃ (boron trichloride), and PCl₅ (phosphorus pentachloride). According to a specific example, a surface region (e.g., a metal or a ceramic surface region with available hydroxide groups) is reacted with silicon tetrachloride as a halogenating agent (in this instance, a chlorosilanization agent). This reaction scheme can be represented, for example, as follows:
M-OH+SiCl₄→M-O—SiCl₃+HCl,
where M corresponds to the metal or ceramic surface. Once they are produced on the surface, the chlorosilane groups are then exposed to a molecule that is reactive with the same (e.g., species comprising hydroxyl groups), thereby forming a covalently coupled molecular species.

The above scheme can be conducted on a wide variety of surfaces, including various metallic and ceramic surfaces, so long as surface hydroxyl groups are available for reaction. This scheme can also be conducted on various metals which form native oxide layers. In this regard, controlled native oxide layers can be formed on most metals used today in medical devices. This technology is well known. The above reaction scheme can also be conducted on surface regions which have been pretreated to establish hydroxyl groups thereon. For example, in some embodiments, a surface region, for example, a polymeric surface region, is pretreated by subjecting it to a glow discharge step. The resulting surface region, which is hydroxylated during the glow discharge step, is then available for reaction in accordance with the above scheme.

Ligand attachment need not be covalent. For example, it is known from Michel R., et al., “Self-organized molecular assembly: patterning of surfaces at the micro- and nano-scale for biological applications,” Langmuir, 2002, 18, 3281-3287, that alkane phosphates from aqueous solution will adsorb onto certain metal oxides, such as titanium or niobium oxide. Due to the presence of titanium oxide on nickel-titanium alloy surfaces, dodecylphosphate (DDP) is expected to be readily adsorbed by self-assembly from aqueous solutions of its ammonium salt, rendering the titanium oxide surface hydrophobic and hence protein-adsorbing. (In alternative embodiments, a thin layer of pure titanium oxide is formed at the nanoparticle surface.) Subsequently, polypeptide containing molecules, including proteins, are adsorbed to the surface.

If desired, ligands can be coupled to only a portion of the nanoparticle surface. For example, lithographic masking techniques can be used to prevent contact with certain portions of the nanoparticles. As another example, see also, for example, A K Salem et al. “Multifunctional nanorods for gene delivery,” Nature Materials, Vol. 2, 2003, pp 668-671, which describes a non-viral gene-delivery system based on multisegment bimetallic nanorods (Au/Ni) that can simultaneously bind compacted DNA plasmids and targeting ligands in a spatially defined manner.

Using the above and other techniques, a wide variety of ligands can be adsorbed or covalently coupled to a wide range of nanoparticle potions. For more information on ligand coupling, see, for example, Mohammed Aslam PhD and Alastair H. Dent, Bioconjugation: Protein Coupling Techniques for the Biomedical Sciences, Nature Publishing Group, 1998; Yuri Lvov et al., Protein Architecture: Interfacing Molecular Assemblies and Immobilization Biotechnology, Marcel Dekker, 1999; and Shtilman, M I, Immobilization on Polymers, VSP International Science Publishers, 1993; the disclosures of which are incorporated by reference.

In many embodiments of the invention, it is desirable to non-invasively image the nanoparticles once they are assembled in vivo. Among currently available non-invasive imaging techniques are included magnetic resonance imaging (MRI), x-ray fluoroscopy and scintigraphic imaging, among others.

Magnetic resonance imaging (MRI) produces images by differentiating detectable magnetic species in the portion of the body being imaged. For contrast-enhanced MRI, it is desirable that the contrast agent have a large magnetic moment, with a relatively long electronic relaxation time. Based upon these criteria, contrast agents such as Gd(III), Mn(II) and Fe(III) have been employed. Gadolinium(III) has the largest magnetic moment among these three and is, therefore, a widely-used paramagnetic species to enhance contrast in MRI. Chelates of paramagnetic ions such as Gd-DTPA (gadolinium ion chelated with the ligand diethylenetriaminepentaacetic acid) have also been employed as MRI contrast agents. In accordance with certain embodiments of the invention, paramagnetic ion chelates can be attached to selected nanoparticle portions using coupling techniques such as those described above.

As seen above, many species useful as tissue targeting ligands are currently available in radiolabeled form, allowing them to be imaged. Alternatively, techniques are well-known for providing ligands with radiolabeled atoms.

With respect to x-ray based fluoroscopy, some nanoparticles such as metallic nanoparticles are inherently more absorptive of x-rays than surrounding tissue. Alternatively, the nanoparticles of the present invention can be provided with contrast agents, in certain embodiments, such as metals (e.g., tungsten, platinum, tantalum, iridium, gold, or other dense metal), metal compounds (e.g., barium sulfate, bismuth subcarbonate, bismuth trioxide, bismuth oxychloride, etc.) or iodinated compounds (e.g., iopamidol, iothalamate sodium, iodomide sodium).

Ultrasound uses high frequency sound waves to create an image of living tissue. A sound signal is sent out, and the reflected ultrasonic energy, or “echoes,” used to create the image. Ultrasound imaging contrast agents are materials that enhance the image produced by ultrasound equipment. Ultrasonic imaging contrast agents introduced into the compositions of the present invention can be, for example, echogenic (i.e., materials that result in an increase in the reflected ultrasonic energy) or echolucent (i.e., materials that result in a decrease in the reflected ultrasonic energy). Suitable ultrasonic imaging contrast agents for use in connection with the present invention include solid particles ranging from about 0.01 to 50 microns in largest dimension (e.g., the nanoparticles of the present invention may provide sufficient contrast in some instances). In other embodiments, nanobubbles (e.g., air filled nanocapsules) are used.

Hence, using the above and other techniques known to those of ordinary skill in the art, nanoparticles can be fabricated and subsequently injected into the vasculature where they attach to diseased or abnormal structures that have an identifiable marker, which may appear, for example, on the endothelium, on exposed basement membrane, on exposed extracellular matrix, and so forth. Further particles then self-assemble into a structure over the particles that initially attach to the tissue. The shape of the endovascular structure assembled will depend, for example, on the shape of the particles, the locations of the ligands, and so forth.

In some embodiments, the self-assembled structures act as stabilizing or isolating structures over diseased or aberrant tissue. For example, in some embodiments, vulnerable plaque (i.e., plaque at risk for rupture) is stabilized by the self-assembly of what effectively amounts to a patch over the plaque. Additionally, if the nanoparticles have the property that they can partition into the plaque (e.g. because they have lipophilic character that carbon nanotubes, among other particles, may possess) they may self assemble in the plaque, which may, for example, allow the assembly to be better retained, stabilize the plaque by the presence of a large structure, and/or release a variety of therapeutic agents (e.g., anti-inflammatory agents to mitigate the processes that cause the plaque to become unstable, agents to enhance healing of the plaque, agents and polymeric precursors that can gel the components of the plaque for stabilization, for instance, by crosslinking of the polymeric precursors upon release, and so forth). Furthermore, the self assembled structure may be used as a diagnostic to locate the position of vulnerable plaques. Depending on the nature of the nanoparticles and their components, they could be visible by MRI (e.g., by using paramagnetic particles) or by catheters with spectroscopic (e.g. near infrared) detectors. For an example of the latter technique, see, e.g., P W Barone, et al. “Near-infrared optical sensors based on single-walled carbon nanotubes,” Nature Materials 4 (2005) 86-92.

In some embodiments, the self-assembled structures perform a mechanical function. For instance, the structures can contract and/or expand upon activation (e.g., by exploiting shape memory or other shape-change properties of the individual particles). Triggers for activating the shape change properties of the material include ultrasound, radiofrequency radiation, microwave radiation, or oscillating magnetic fields as discussed above. Further, a molecular aggregate with an interparticle binding ligand and a further ligand may be used to cause a conformation change when an injected agent or an agent that circulates in blood binds to this further ligand. For example, various molecules are known which change in conformation upon binding to other agents. Molecules are also known which change in conformation upon exposure to energy, which causes partial or full denaturation.

Using such techniques, in the case where a structure is self-assembled over obstructing plaque or restenotic structures, the structure is then expanded to increase the vessel diameter by activating the shape memory property of the self-assembled particles. In this instance, the self-assembled structure is acting as an expanded stent segment. In one specific embodiment, a U-shaped shape memory rod is employed as the nanoparticle portion and ligands are provided at the ends of the U. These particles then undergo shape change and open up when triggered (e.g., by heating).

This shape change can also be used to impose a force for shrinking damaged and dilated tissue. A specific example of a beneficial shrinking structure is the case where an adherent structure is self-assembled over scarred heart muscle (e.g. from an old infarct), and then activated to contract (e.g., linear shape memory rods are employed which become U-shaped upon triggering). This contraction reshapes the heart, reducing the ventricular volume, increasing ejection fraction, and leading to positive remodelling of the heart. The reduced volume increases the force of heart contraction and ejection fraction consistent with Starling's law of the force of heart contraction. This concept is practiced on gross scale by surgical interventions by removing the heart muscle, by ventricular reduction using the Battista or Dorr procedures, or by shrinking the scarred tissue and patching using processes such as those available from Hearten Medical, Irvine, Calif., USA.

Self assembled structures can also be triggered, using techniques such as those discussed above, to release drugs or other beneficial agents that are contained in or attached to the nanoparticles. For example, these agents can be antirestenosis agents in order to treat plaque. As another example, these agents can correspond to components of single- or multi-component adhesives or glues (e.g., fibrinogen, thrombin, cyanoacrylate adhesive, etc.) to further stabilize vulnerable plaque and for aneurysmal management. As yet another example, these agents can correspond to growth factors to repair vascular tissue or to revascularize injured and/or scarred tissue, such as heart muscle following infarct.

In further embodiments, the self-assembling compositions of the present invention are used to target diseased or infected tissue, including tissue infected with bioterror agents. Upon activation the self-assembled structures (most likely in capillary beds), a drug or other argent is released to treat the disease or infection.

As a specific example, inhalation of infectious agents into the lungs is expected to result in changes of the endothelium of the capillary beds in the lungs. These aberrant endothelium can be targeted for the formation of self-assembled structures that will release anti-infectious agents at only the local site of infection. For instance, as noted above, autologous leukocytes concentrate at inflammatory and infectious sites, as do cytokines, chemokines, complement factors, chemotactic peptides, other chemotactic factors, as well as antagonists to the tuftsin receptor. Hence, ligands for nanoparticles that are intended for delivery at sites of local infection can be selected from these species. The above and other techniques could allow the use of relatively toxic agents, since they are only released locally at the site of infection.

Self assembled endovascular structures in accordance with the present invention can also be used as a scaffolds for tissue repair. These structures would contain ligands that bind endogenous cells or injected cells. For example, as indicated above, peptides having affinity for the alpha(v)beta(3) integrin can be used to target areas of vascular damage. Moreover, integrins can also be used to target areas of vascular damage, as they recognize a wide variety of extracellular matrix components and cell-surface receptors, as previously noted. Preferred nanoparticles for forming self assembled scaffolds for tissue repair include nanoparticles of extracellular matrix materials such as collagen (e.g., type IV collagen), glycosaminoglycans, synthetic particles providing a coating with ECM-like materials to encourage healing, and so forth. The self assembled nanoparticles can also be provided with drugs or other agents which are released to attract and/or promote growth of the desired endogenous cell type.

Although various embodiments are specifically illustrated and described herein, it will be appreciated that modifications and variations of the present invention are covered by the above teachings and are within the purview of the appended claims without departing from the spirit and intended scope of the invention.

1. An injectable composition comprising self-assembling nanoparticles, said self-assembling nanoparticles comprising: (a) a nanoparticle portion, (b) tissue binding ligands attached to the nanoparticle portion that cause preferential binding and accumulation of the nanoparticles at one or more targeted tissue locations upon injection of the composition into the body, and (c) first and second interparticle binding ligands attached to the nanoparticle, which result in interparticle binding at the one or more targeted tissue locations.

2. The composition of claim 1, wherein at least one of the first and second interparticle binding ligands is activated in vivo at said one or more targeted tissue locations.

3. The composition of claim 1, wherein said nanoparticle portions are selected from spherical nanoparticle portions, plate-shaped nanoparticle portions, and elongated nanoparticle portions.

4. The composition of claim 1, wherein said nanoparticle portions are inorganic nanoparticle portions.

5. The composition of claim 4, wherein said inorganic nanoparticle portions are metallic nanoparticle portions.

6. The composition of claim 1, wherein said nanoparticle portions are organic nanoparticle portions.

7. The composition of claim 6, wherein said organic nanoparticle portions are polymeric nanoparticle portions.

8. The composition of claim 1, wherein the shape of said nanoparticle portions can be changed in vivo by administering a triggering procedure.

9. The composition of claim 8, wherein said nanoparticle portions have a shape memory.

10. The composition of claim 9, wherein said nanoparticle portions are formed from shape memory metals or shape memory polymers.

11. The composition of claim 9, wherein said nanoparticle portions expand upon triggering said shape memory.

12. The composition of claim 9, wherein said nanoparticle portions contract upon triggering said shape memory.

13. The composition of claim 8, wherein said nanoparticle portions are formed from a heat-shrinkable material.

14. The composition of claim 1, wherein said self-assembling nanoparticles comprise a releasable adhesive species.

15. The composition of claim 1, wherein said self-assembling nanoparticles comprise a drug that is released in vivo and subsequent to nanoparticle self-assembly.

16. The composition of claim 15, wherein said drug is covalently coupled to said nanoparticle portion.

17. The composition of claim 15, wherein said drug is encapsulated and attached to said nanoparticle portion.

18. The composition of claim 15, wherein said nanoparticle portion comprises a drug.

19. The composition of claim 15, wherein said nanoparticle portion comprises an encapsulated drug.

20. The composition of claim 15, wherein said drug is selected from an anti-restenosis drug, an anti-thrombotic drug, growth factors, an anti-inflammatory drug, cell adhesion proteins, and combinations of the same.

21. The composition of claim 1, wherein said self-assembling nanoparticles comprise magnetic nanoparticles.

22. The composition of claim 1, wherein said tissue binding ligands are selected from antibodies, integrins, cell receptor mimetics and combinations of the same.

23. The composition of claim 1, wherein said first and second interparticle binding ligands comprise antibody-antigen pairs.

24. A kit for self-assembly of nanoparticles in vivo comprising:
(a) a first injectable composition comprising first self-assembling nanoparticles that comprise (i) a first nanoparticle portion (ii) tissue binding ligands attached to the first nanoparticle portion that cause preferential binding and accumulation of the first self-assembling nanoparticles at one or more targeted tissue locations upon injection of the first composition into the body, and (iii) first interparticle binding ligands attached to the first nanoparticle portion to promote interparticle binding; and
(b) a second injectable composition comprising second self-assembling nanoparticles which comprise (i) a second nanoparticle portion and (ii) second interparticle binding ligands attached to the second nanoparticle portion that preferentially bind to the first interparticle binding ligands of the first nanoparticles upon injection of the second composition into the body,
wherein said first and second nanoparticle portions can be formed from the same or different materials.

25. The kit of claim 4, wherein said second self-assembling nanoparticles further comprise tissue binding ligands attached to the second nanoparticle portion that cause preferential binding of the second nanoparticles to tissue at said one or more target locations upon injection into the body.

26. The kit of claim 4, wherein said second self-assembling nanoparticles do not further comprise tissue binding ligands attached to the second nanoparticle portion.

27. The kit of claim 4, further comprising a third injectable composition comprising third self-assembling nanoparticles which comprise the following: (i) a third nanoparticle portion and (ii) third interparticle binding ligands attached to the third nanoparticle portion that preferentially bind to the second interparticle binding ligands of the second nanoparticles upon injection into the body, wherein said third self-assembling nanoparticles do not further comprise tissue binding ligands attached to the third nanoparticle portion, wherein said first, second and third nanoparticle portions can be formed from the same or different materials, and wherein said first and third interparticle binding ligands can be the same or different.

28. The composition of claim 6, wherein said organic nanoparticle portions comprise protein.
